(19) United States
(12) Patent Application Publication
(10) Pub. No.: US 2021/0132053 A1
(43) Pub. Date: May 6, 2021

(54) IMAGING ASSAYS

(71) Applicant: Oxford Nanoimaging Limited, Oxford, Oxfordshire (GB)

(72) Inventors: Stanley Dinesh CHANDRADOSS, Oxford (GB); Bo JING, Oxford (GB); Mengqiu LI, Oxford (GB)

(73) Assignee: Oxford Nanoimaging Limited, Oxford, Oxfordshire (GB)

(21) Appl. No.: 17/049,551

(22) PCT Filed: May 7, 2019
(86) PCT No.: PCT/GB2019/051251

§ 371 Date: Oct. 21, 2020

(30) Foreign Application Priority Data
May 4, 2018 (GB) 1807409.6

(51) Int. Cl. G01N 33/543; G01N 33/542

(52) U.S. Cl. CPC G01N 33/54353; G01N 33/54393; G01N 33/542

(57) ABSTRACT

The present application relates to assays and systems for the detection of analyte molecules in a liquid sample, preferably a biological sample. In particular the invention relates to a method for determining the presence of a target analyte in a liquid sample, comprising contacting and incubating the sample with a first proximity probe comprising an analyte-binding domain having specificity for the target analyte, wherein the first proximity probe is tethered to a solid support by a polymeric or biopolymeric tether molecule which alters the observed properties.

The present application relates to assays and systems for the detection of analyte molecules in a liquid sample, preferably a biological sample.

Antibody-based analytical techniques are popular and well-known analytical techniques which have been widely adopted in biochemistry and medicine for the qualitative and quantitative detection of analytes in liquid samples. The most common type of antibody-based assay is the enzyme-linked immunosorbent assay, or “ELISA”. A variety of different ELISA techniques are known, all of which fundamentally rely upon the specific binding of an enzyme-labelled antibody to an antigen.

Due to the high specificity of antibody-antigen interactions, ELISA techniques are renowned for their high sensitivity and ability to detect antigens at low concentrations. This sensitivity has led to the widespread adoption of ELISA in applications such as detection of the presence of antibodies in serum samples to determine the presence or absence of a disease in a patient; the analysis of food samples to determine the presence of allergens; toxicology screening; and the detection of biomarkers such as proteins which signify the presence of particular types of tumour.

It is nevertheless desirable to improve the sensitivity of ELISA-based techniques further in order to improve their utility in the early diagnosis of diseases. At very early stages of a disease the level of biomarkers present in body fluid samples may be below the level of detection (LOD) of current assay techniques and therefore such biomarkers may not be detected, or may be detected only at low levels which do not permit a confident diagnosis to be made. Low levels of biomarkers may also be present in the case of some non-early-stage diseases, giving rise to the same problems of detection and diagnosis. However, it is desirable to be able to make diagnoses as early and as confidently as possible, particularly in the case of diseases such as cancer, neurodegenerative diseases and infections, in which the prognosis can be significantly improved by early detection and treatment.

As an example, a small early-stage cancer tumour having a volume of about 1 mm³ typically consists of approximately 1 million cells. The average adult human has a blood volume of about 5 litres. Assuming that each cell in the tumour secretes about 5000 protein molecules which act as cancer biomarkers into 5 litres of circulating blood, the concentration of cancer biomarker molecules in the blood will be approximately 2×10⁻¹⁵ mol dm⁻³ (i.e. approximately 2 femtomolar). This is below the sensitivity of many current assay techniques such as ELISA or surface plasmon resonance (sensitivity typically of the order of pM-nM).

The LOD can be formally defined as the concentration of analyte when the signal is extrapolated to the background level (at zero concentration of analyte), plus 3 standard deviations of the background level. To obtain a low LOD, it is clear that both the background level and its variation must be minimised.

The LOD can be also defined as the concentration corresponding to the signal level which is the average signal level of the blank sample plus the standard deviation of the blank sample multiplied by 3. The definition is shown below, where “f” is the calibration function that converts raw signal level to concentration. The calibration function is determined experimentally using standard solutions containing known concentrations of the analyte of interest. LOD=f (blank average+3×blank standard deviation)

Recently, ELISA-based techniques have been introduced to the market which are advertised as having femtomolar or sub-femtomolar LODs. For example, “Simoa”® assays and instruments available from the Quanterix Corporation (Lexington, Mass., USA) have an advertised LOD which is subfemtomolar. The SMC™/Erenna® assay technology available from Singulex, Inc. (Alameda, Calif., USA) also has an advertised LOD which is subfemtomolar. However, such techniques nevertheless suffer from drawbacks which it is desirable to overcome. For example, the “Simoa” techniques have a limited dynamic range and do not give quantitative results at analyte concentrations above about 10⁻¹⁴ M. “SMC”/“Erenna” techniques detect single, diffusing, fluorophore-labelled reported antibodies which are dissociated from the antigen. Variation in the stoichiometry of antibody-antigen binding, antibodies sticking to the reaction chambers, and inherent stochasticity of diffusing molecules introduce significant margins of uncertainty to the quantification of antigens detected using such techniques. Both Simoa and SMC/Erenna techniques employ costly and large (˜1.5-3 m³) instruments which require significant lab space. Further, despite the fact that ELISA protocols conventionally employ a blocking buffer which is intended to prevent non-specific binding of reporter antibodies to the solid support, a certain degree of non-specific binding will inevitably take place even in highly-sensitive assays such as Simoa, SMC or Erenna assays. This non-specific binding gives rise to a background signal which cannot be distinguished from the signals obtained from antibodies which are specifically bound to a target analyte. These background signals constrain the LOD and limit the ability of conventional ELISA-based techniques to provide accurate quantification of analyte concentrations.

An additional factor constraining the applicability of ELISA-based techniques, in particular in the field of medical diagnostics, is that the volumes of sample required can be of the order of millilitres. Where the sample to be analysed is a body fluid sample (e.g. a blood or cerebrospinal fluid sample) this can require the extraction of large volumes from a patient, which can be an unpleasant experience. It is therefore desirable to develop assay techniques requiring lower sample volumes.

Another feature of conventional ELISA-based techniques is that the time taken to perform an assay and analyse the results is often in the region of 2 to 3 hours or longer. It is therefore desirable to develop assay techniques having sub-1-hour run times. This will not only increase the turnover rate for performing analysis of large batches of samples, but will also open the way to genuine point-of-care testing employing ELISA-based assays.

As an alternative to fluorescence based assays, measurements using light scattering have also been used to detect the presence of biomolecules. For example Interferometric scattering microscopy (iSCAT) is an optical microscopy technique that relies on illuminating a scattering object and collecting the interference between the light field scattered by the scattering object and a reference light field, provided by the reflection of the illumination at the interface near where the scattering object is located. In the absence of the scattering object, the light field detected on the camera is purely from the reflected light field. In the presence of the scattering object, the intensity of the reflected light is attenuated by the interference between scattered light from the object and the reflected light. iSCAT has been applied to a number of biomolecule detection applications where the mass of the biomolecule is sufficiently large to produce a sufficiently strong scattering light field by itself, or where the biomolecule is labelled by a nanoparticle with higher mass.

The present inventors have now surprisingly found that the assays according to the invention are capable of distinguishing between specific and nonspecific binding of reporter antibodies, thereby improving the LOD to a typical level of 10⁻¹⁵ M or below, with 10⁻¹⁸ M or below being achievable in at least some cases. The assays of the invention can be performed on sample volumes of 10 μl or less, compatible with the volume of a blood sample obtained by a “finger prick” skin puncture method. The assays of the invention are capable of quantifying the number of molecules of analyte to a high degree of accuracy even when only a few tens of analyte molecules are present in the sample volume. The assays of the invention typically have a run-time of less than 1 hour.

### SUMMARY OF THE INVENTION

The present invention relates to the imaging of individual marker molecules and uses a tether molecule to attach single markers to a solid support. The tether molecule is sufficiently long such that material attached via the tether has different characteristics, such as the range of movement and the velocity of movement, to material attached non-specifically to the surface without the tether. Thus the correctly tethered material can be differentiated from a potentially higher level of background signals which are either immobilised not via the tether or are not immobilised.

The present inventors have surprisingly found that by tethering a first proximity probe to a support such that a spaced relationship exists between the first proximity probe and the support, it is possible to distinguish between background or “false positive” signals and genuine signals arising from interactions between the first proximity probes and fluorescently labelled products in solution. Due to the length of the tether, the fluorescent probes attached via the tether appear to have a larger area of motion (i.e. greater diffusion) than the molecules which are merely randomly adhered to the surface.

The present invention therefore provides a method for determining the presence of a target analyte in a liquid sample, comprising the steps of:
(i) contacting and incubating the sample with a first proximity probe comprising an analyte-binding domain having specificity for the target analyte, wherein the first proximity probe is tethered to a solid support by a polymeric or biopolymeric tether molecule, thereby tethering the target analyte to the support;
(ii) generating signals from individual molecules which are specific to the tethered target analyte; and
(iii) detecting the tethered target analyte by observing the motion of the signals on the solid support.

The assay detects the signals from molecules attached to individual tethers and the motion thereof. Whilst optionally more than one target analyte can be attached to each tether, and each target analyte can optionally be labelled with multiple fluorophores or other labels, the technique follows the motion of individual signal generating species. The signal generating species will generally consist of individual target analytes, which may be multiply labelled.

The signals can be fluorescent signals. The target analyte can be labelled via a binding probe such as a fluorescently labelled antibody or nucleic acid probe, either before or after immobilisation. Alternatively, the binding probe can be coupled to an enzyme which initiates a reaction that generates a fluorescent product. The nature of the tether affects the fluorescent signal observed.

The signals can be generated by light scattering, in which can the target analyte can be unlabeled. The target analyte can be labelled with a binding probe which in turn is coupled to one or more nanoparticles in order to increase the mass of the marker species thereby increasing the scattered light.

The tether molecule optionally has a mean end-to-end distance of about 50 nm to about 1000 nm in aqueous solution. The presence of the tether allows the signal generating molecule to move within a larger range around the anchoring point of the tether on the surface compared to signal generators bound directly to the surface. The detection can be performed either by tracking the motion over time using a series of images or by taking a single image that shows the area of the surface over with the signal can travel.

### DETAILED DESCRIPTION

The present invention uses a molecular tether bound to solid support to capture analytes for single molecule imaging and detection. The binding point of the analyte on the tether is sufficiently far from the surface that the attached biomolecule has distinct motion characteristics which are measured using a variety of methods, such as fluorescence microscopy imaging or iSCAT, after the analyte has been labelled with appropriate binding probes which give rise to fluorescence signals or scattering signals.

The present invention therefore provides a method for determining the presence of a target analyte in a liquid sample comprising the steps of:
(i) contacting and incubating the sample with a first proximity probe comprising an analyte-binding domain having specificity for the target analyte, wherein the first proximity probe is tethered to a solid support by a polymeric or biopolymeric molecule(s);
(ii) contacting and incubating the sample with a second proximity probe comprising an analyte-binding domain having specificity for the target analyte, wherein
   a. the second proximity probe comprises a fluorophore; or
   b. the second proximity probe is conjugated to a reporter enzyme prior to contacting the sample; or
   c. the second proximity probe is conjugated to a reporter enzyme simultaneously with or after contacting the sample; or
   d. the second proximity probe comprises an oligonucleotide sequence for fluorescence in-situ hybridisation (FISH); or
   e. the second proximity probe is conjugated to a nanoparticle
(iii) a. if the second proximity probe is conjugated to a reporter enzyme, adding a fluorogenic substrate of the reporter enzyme to the sample to generate a fluorescent reaction product; or
   b. if the second proximity probe comprises an oligonucleotide sequence for fluorescence in-situ hybridisation (FISH), hybridising the second proximity probe with a FISH probe which comprises a fluorophore and an oligonucleotide sequence complementary to the oligonucleotide sequence of the second proximity probe;
(iv) illuminating the sample to cause the fluorophore or fluorescent reaction product to fluoresce or cause the nanoparticle to scatter light; and
(v) detecting the target analyte by observing the motion of the fluorescence or scattering signal from the fluorophore or fluorescent reaction product or nanoparticle.

In all embodiments of the invention, the steps of contacting and incubating the sample with the first proximity probe and contacting and incubating the sample with the second proximity probe may be performed sequentially (step (i) followed by step (ii) or step (ii) followed by step (i)) or simultaneously. Preferably, however, they are performed sequentially and particularly preferably with step (i) followed by step (ii). Optionally, steps (i) and (ii) are performed sequentially and a washing step is performed in between steps (i) and (ii). Any suitable washing buffer may be employed for the washing step. The washing step helps to remove molecules from the sample which have not been bound by the first proximity probe or non-specifically bound to the support, thereby helping to minimise any background fluorescence or light scatter.

Step (iii) (where present) and steps (iv) and (v) are performed after steps (i) and (ii). optionally a further washing step is performed between steps (ii) and (iii) (where (iii) is present) or between steps (ii) and (iv) (where step (iii) is absent) in order to remove any unbound molecules of the second proximity probe. Steps (iv) and (v) are performed after step (iii) (where present) or after step (ii) (where step (iii) is absent). Steps (iv) and (v) are performed simultaneously with each other.

The term “detecting” as used herein includes both qualitative and quantitative measurements of a target analyte. In one aspect, the method of the invention is used to identify the mere presence of the analyte in a liquid sample. In another aspect the method of the invention is used to test whether the analyte is at a detectable level. In a further aspect, the method of the invention is used to quantify the amount of analyte present in the sample. In another aspect, the method of the invention is used to quantify the amount of analyte in the sample and further to compare the amounts of analyte in different samples.

In certain embodiments of the invention the liquid sample comprises, consists essentially of or consists of a body fluid from a human or non-human animal subject, preferably from a human subject. Preferred body fluids include saliva, cerebrospinal fluid, vitreous fluid, lymph fluid, synovial fluid, follicular fluid, seminal fluid, amniotic fluid, milk, perspiration, tears, urine and blood. Preferably the liquid sample is a blood sample from a human subject. The blood sample may be a whole blood sample, a plasma sample or a serum sample.

In other embodiments of the invention the liquid sample comprises, consists essentially of or consists of a fluid from a plant, for example xylem sap, phloem sap, cell sap, or cytosol. The invention may thus be used for applications such as detecting plant diseases, plant hormones, etc.

The target analyte is preferably a biomarker indicating the presence of a disease such as cancer or a neurodegenerative disease, or indicating the presence of an infection. A typical biomarker may for example be a hormone, a (poly)peptide (e.g. a protein or enzyme), carbohydrate, antibody, or oligonucleotide or small molecule biomarker such as folic acid. Preferably the analyte (e.g. the biomarker as described above) is an antigen and/or a nucleic acid sequence.

The first proximity probe may also be referred to herein as a “capture probe” and the second proximity probe may also be referred to herein as a “reporter probe”.

The first proximity probe may comprise or consist of a protein, an antibody, lectin, soluble cell surface receptor, combinatorially derived protein from phage display or ribosome display, carbohydrate, aptamer, affimer, affibody, affilin, affitin, alphabody, anticalin, avimer, DARPin, monobody, oligonucleotide or polynucleotide, or combinations thereof. Preferably the first proximity probe comprises or consists of an antibody.

The term “antibody” as used herein is used in the broadest conventional sense and includes intact monoclonal antibodies, intact polyclonal antibodies, multispecific antibodies (e.g. bispecific antibodies) formed from at least two intact antibodies, chimeric antibodies, single-domain antibodies (also called “nanobodies”) and antibody fragments, so long as they exhibit the desired affinity for the target analyte. An “intact antibody” is one comprising heavy- and light-chain variable domains as well as an Fc region. “Antibody fragments” comprise a portion of an intact antibody, preferably comprising the antigen-binding or variable region thereof. Examples of antibody fragments include Fab, Fab′, F(ab′)₂, and Fv fragments; diabodies; linear antibodies; single-chain antibody molecules; and multispecific antibodies formed from antibody fragments.

The first proximity probe is tethered to a solid support. Tethering of the first proximity probe to the support takes place prior to, simultaneously with or after contacting and incubating the sample with the first proximity probe. Tethering of the first proximity probe to the support may also take place simultaneously with or after the addition and incubation of the second proximity probe or simultaneously with or after conjugation of the reporter enzyme. The first proximity probe remains tethered to the support throughout all subsequent steps of the method following its tethering to the support.

The tether molecule which tethers the first proximity probe to the support (which may be referred to as a “tether” or “tether molecule”) may be a polymer or a biopolymer. Preferably the tether molecule should have a mean end-to-end distance of from about 50 nm to about 1500 nm in aqueous solution, such as about 150 nm to about 1360 nm, about 250 nm to about 650 nm, about 350 nm to about 550 nm or about 450 to about 550 nm. For fluorescence measurements, preferably the tether molecule should have a mean end-to-end distance of from about 50 nm to about 250 nm in aqueous solution, e.g. from about 50 nm to about 175 nm, about 75 nm to about 150 nm or about 100 nm to about 200 nm. For scattering measurements the tether can be longer.

Suitable tether molecules include surfactants, lipids, peptides, proteins, oligosaccharides, polysaccharides, oligonucleotides and polynucleotides, such as DNA or RNA. Where DNA or RNA is employed this may be double-stranded (dsDNA or dsRNA or a DNA-RNA double stranded hybrid) or single-stranded and may be naturally-occurring or synthetic. DNA origami structures may also be employed as tether molecules. Where DNA origami structures are employed, these may have a plurality of binding sites for proximity probes. Thus in certain embodiments of the invention the tether molecule is dsDNA or dsRNA having a mean end-to-end distance of from about 50 nm to about 250 nm in aqueous solution, e.g. from about 50 nm to about 175 nm, about 75 nm to about 150 nm or about 100 nm to about 200 nm. In practice a plurality of tether molecules will normally be employed. These will typically be the same as one another and have the same mean end-to-end distance as one another. However, in some embodiments a plurality of tether molecule species may be employed simultaneously which have different mean end-to-end distances compared to one another. Each tether species can be coupled to a distinct species of capture probes detecting distinct species of analytes, allowing simultaneous, multiplexed analyte detection by measuring the motion of the analytes on differently sized tethers.

Where double-stranded DNA or RNA is employed as a tether it is particularly preferred that it should have a length of between about 450 and about 4000 base pairs, preferably between about 500 and 3000 base pairs, between about 500 and about 2000 base pairs, between about 750 and about 1600 base pairs, particularly between about 800 and about 1300 base pairs, between about 800 and about 1000 base pairs, or between about 900 and about 1100 base pairs, such as about 1000±100 base pairs or about 1000 base pairs.

Double-stranded DNA is preferred for use as a tether as the mechanical properties and dynamics of dsDNA are well-understood as described in, for example, Yin et al., “Tethered Particle Motion Method for Studying Transcript Elongation by a Single RNA Polymerase Molecule”, Biophys. J. 1994, Dec. 1; 67(6):2468-78, or May et al., “Tethered fluorophore motion: studying large DNA conformational changes by single-fluorophore imaging”, Biophys. J. 2014 Sep. 2; 107(5):1205-1216.

In some embodiments both the tether and the first proximity probe may each comprise or consist of an oligonucleotide or polynucleotide. In such embodiments the tether and the first proximity probe may optionally be synthesised together.

In certain embodiments of the invention the solid support is coated with a metal film (for instance 20 nm of gold evaporated onto the solid support) which quenches the fluorescence from fluorophores within about 50 nm of the metal film. The fluorescence signals arising from target analyte molecules or proximity probes which bind non-specifically in proximity to the surface are efficiently quenched, whereas fluorescence from those molecules on tethers which are further away from the surface are not affected.

In certain embodiments of the invention the solid support is passivated. Passivation of the surface helps to reduce nonspecific binding of analytes, proximity probes and fluorescent molecules to the surface of the support which could negatively impact upon the accuracy or sensitivity of detection. Passivation may take place before the tether molecule is bound to the solid support. Passivation may be performed using any conventional blocking buffer known to the skilled person for use in ELISA assays or single-molecule TIRF experimentation, for example. Molecules from the blocking buffer (passivating molecules) adsorb to the support and reduce the number of available sites for nonspecific binding.

The tether molecule is attached (tethered) to the solid support. The tether molecule may be bound directly (e.g. via a covalent bond) to the solid support. Preferably, however, the tether molecule may be bound indirectly to the solid support, by binding the tether molecule specifically to a molecule which is adsorbed or conjugated to the support. Preferably the molecule to which the tether molecule binds specifically is a molecule from the blocking buffer which is adsorbed or conjugated to the surface of the support and provides specific binding sites for the tether.

The support is preferably passivated with an aqueous solution comprising a protein (e.g. bovine serum albumin (BSA) or human serum albumin (HSA))), a nonionic surfactant (such as polysorbate 20 (e.g. Tween®-20), Triton X-100, or poloxamer 407 (e.g. Pluronic™ F127)), polyethylene glycol (PEG) (optionally in the form of an ester), or any mixture thereof. Such proteins, surfactants, PEG and mixtures thereof may be referred to as “passivation/passivating molecules” or “passivation/passivating agents”. The use of passivating agents is particularly preferable as the tether molecule can then be specifically bound to the adsorbed passivating agents in order to connect the tether to the solid support. Optionally, some or all of these passivation molecules may be conjugated with a biotin group, or a fluorescein isothiocyanate (FITC) group, or a digoxigenin (DIG) group, or other molecules which are part of common molecular interaction toolkits known to the skilled person.

The tether molecule may be bound to the adsorbed passivating molecule or directly to the surface via a covalent or noncovalent interaction, e.g. by virtue of complementary interactions between moieties on the tether molecules and passivating molecules on the surface or by virtue of interactions between moieties on the tether molecules and the surface itself. Suitable interactions for this purpose include biotin-streptavidin interactions, biotin-avidin interactions, streptag-strep-tactin interactions, Fluorescein Isothiocyanate (FITC)-Anti FITC ab interactions, Digoxigenin (DIG)-Anti DIG ab interactions, Nickel-NTA interactions, Copper-NTA interactions, Maleimide group-sulfhydryl group interactions, N-hydroxysuccinimide (NHS) ester-amine interactions, thiol-thiol interactions or alkyne-azide interactions (including, but not limited to, copper-catalysed alkyne-azide cycloaddition (CuAAC), ruthenium-catalysed alkyne-azide cycloaddition (RuAAC), strain-promoted alkyne-azide cycloaddition (SPAAC)), aldehyde-amine interactions (optionally followed by amine reduction), aldehyde-hydrazine interactions, amine-tetrazine interactions, Staudinger ligation. Biotin-avidin-biotin or biotin-streptavidin-biotin interactions may also be employed in which both the tether and the passivating molecule bear a biotin moiety and avidin or streptavidin is employed to bridge the two biotin moieties together.

Some molecules can act as a passivation agent and tether molecule at the same time. Long chained surfactant molecules such as poloxamers (e.g. Pluronic F-127) are particularly suitable for such purposes. Such surfactant molecules may in some cases require functionalisation in order to improve their binding ability to the support. Suitable functionalisations for such purposes may be determined readily by the skilled person. Where tether molecules having an innate passivating ability are employed, it is not typically necessary to passivate the support prior to attaching the tether molecules. In such instances it is therefore preferable that the surface is not passivated other than by the tether molecule.

The first proximity probe can be bound to the tether molecule via a covalent or non-covalent interaction. Suitable interactions for this purpose include biotin-streptavidin interactions, biotin-avidin interactions, streptag-strep-tactin interactions, Fluorescein Isothiocyanate (FITC)-Anti FITC ab interactions, Digoxigenin (DIG)-Anti DIG ab interactions, Nickel-NTA interactions, Copper-NTA interactions, Maleimide group-sulfhydryl group interactions, thiol-thiol interactions, NHS-ester-amine interactions or alkyne-azide interactions (including, but not limited to, copper-catalysed alkyne-azide cycloaddition (CuAAC), ruthenium-catalysed alkyne-azide cycloaddition (RuAAC), strain-promoted alkyne-azide cycloaddition (SPAAC)), aldehyde-amine interactions (optionally followed by imine reduction), aldehyde-hydrazine interactions, amine-tetrazine interactions, Staudinger ligation. Biotin-avidin-biotin or biotin-streptavidin-biotin interactions may also be employed in which both the tether and the passivating molecule bear a biotin moiety and avidin or streptavidin is employed to bridge the two biotin moieties together.

In one embodiment both the first proximity probe and the tether molecule are biotinylated and the first proximity probe is bound to the tether molecule via a biotin-streptavidin, biotin-avidin, biotin-streptavidin-biotin or biotin-avidin-biotin interaction, and the tether molecule is bound to a passivating molecule via a biotin-streptavidin, biotin-avidin, biotin-streptavidin-biotin, or biotin-avidin-biotin interaction.

Preferably the interactions which (directly or indirectly) bind the tether molecule to the surface and which bind the tether molecule to the first proximity probe are each independently selected from biotin-streptavidin interactions, biotin-avidin interactions, biotin-streptavidin-biotin interactions, biotin-avidin-biotin interactions, streptag-strep-tactin interactions, Fluorescein Isothiocyanate (FITC)-Anti FITC ab interactions, Digoxigenin (DIG)-Anti DIG ab interactions, Nickel-NTA interactions, Copper-NTA interactions, Maleimide group-sulfhydryl group interactions, thiol-disulfide interactions, NHS-ester-amine interactions or Alkyne-azide interactions, provided that the type of interaction binding the tether molecule to the surface is different from the type of interaction binding the tether molecule to the first proximity probe. In such embodiments the tether molecule is preferably dsDNA.

The second proximity probe may (independently of the nature of the first proximity probe) comprise or consist of an antibody as defined above, a lectin, soluble cell surface receptor, combinatorially derived protein from phage display or ribosome display, carbohydrate, aptamer, affimer, affibody, affilin, affitin, alphabody, anticalin, avimer, DARPin, monobody, enzyme, or oligonucleotide.

In some embodiments of the invention the second proximity probe comprises a fluorophore. For example, the second proximity probe may comprise or consist of a fluorophore-labelled antibody, lectin, soluble cell surface receptor, combinatorially derived protein from phage display or ribosome display, carbohydrate, aptamer, affimer, affibody, affilin, affitin, alphabody, anticalin, avimer, DARPin, monobody, enzyme or oligonucleotide. Preferably the second proximity probe comprises or consists of a fluorophore-labelled antibody, enzyme or oligonucleotide. In a particularly preferable embodiment the second proximity probe is a fluorescence in situ hybridization probe (FISH probe) which comprises a fluorophore and an analyte-binding domain which is an oligonucleotide sequence complementary to an oligonucleotide sequence of the target analyte. In such embodiments the FISH probe hybridises with the target analyte when the second proximity probe is contacted and incubated with the sample.

Optionally the fluorophore in any embodiment of the second proximity probe may be a quantum dot, a metallic nanoparticle, or a polymeric nanoparticle, or the composite thereof with fluorescent properties.

Optionally the second proximity probe may carry a nanoparticle which increases the scattered light. The nanoparticle may be quantum dot, a metallic nanoparticle, or a polymeric nanoparticle, or the composite thereof with or without fluorescent properties.

In certain embodiments the invention therefore provides a method for determining the presence of a target analyte in a liquid sample, comprising the steps of:
(i) contacting and incubating the sample with a first proximity probe comprising an analyte-binding domain having specificity for the target analyte, wherein the first proximity probe is tethered to a solid support by a polymeric or biopolymeric tether molecule;
(ii) contacting and incubating the sample with a second proximity probe comprising an analyte-binding domain having specificity for the target analyte, wherein the second proximity probe comprises a fluorophore;
(iii) illuminating the sample to cause the fluorophore to fluoresce; and
(iv) detecting the target analyte by observing fluorescence from the fluorophore.

In certain embodiments the invention therefore provides a method for determining the presence of a target analyte in a liquid sample, comprising the steps of:
(i) contacting and incubating the sample with a first proximity probe comprising an analyte-binding domain having specificity for the target analyte, wherein the first proximity probe is tethered to a solid support by a polymeric or biopolymeric tether molecule;
(ii) contacting and incubating the sample with a second proximity probe comprising an analyte-binding domain having specificity for the target analyte, wherein the second proximity probe comprises a nanoparticle;
(iii) illuminating the sample to cause interference scattering (iSCAT) from the nanoparticle; and
(iv) detecting the target analyte by observing the motion of the nanoparticles.

The tether is sufficiently long that, if a long-exposure image is taken, the attached biomolecule appears over a larger area of the surface compared to a biomolecule attached without the tether; if a movie, or consecutive short-exposure images, is taken, the motion of the molecules can be analysed and a variety of measurements, such as the range of movement, the velocity of movement, diffusion constant, output from machine learning models, can be made and used to distinguish between tethered molecule and other observed molecules.

In such embodiments, steps (i) and (ii) may be performed sequentially or simultaneously. Preferably, however, they are performed sequentially. Optionally, steps (i) and (ii) are performed sequentially and a washing step is performed in between steps (i) and (ii). Any suitable washing buffer may be employed for the washing step. The washing step helps to remove molecules from the sample which have not been bound by the first proximity probe or non-specifically bound to the support, thereby helping to minimise any background fluorescence or light scatter.

In such embodiments, steps (iii) and (iv) are performed after steps (i) and (ii). Preferably a further washing step is performed between steps (ii) and (iii) in order to remove any unbound molecules of the second proximity probe. Steps (iii) and (iv) are performed after step (ii). Steps (iii) and (iv) are performed simultaneously with each other.

In some embodiments, the second proximity probe with a conjugated fluorophore is incubated in the sample containing the analyte and co-contacted with the first proximity probe for detection. The tethered molecules can be distinguished from the untethered molecules without needing to remove the untethered molecules in any wash steps. Therefore described herein is a method for determining the presence of a target analyte in a liquid sample, comprising the steps of:
(i) contacting and incubating the sample with a ‘second’ proximity probe comprising an analyte-binding domain having specificity for the target analyte to bind the second proximity probe to the target analyte in solution;
(ii) contacting and incubating the sample having the bound proximity probe with a ‘first’ proximity probe comprising an analyte-binding domain having specificity for the target analyte, wherein the first proximity probe is tethered to a solid support by a polymeric or biopolymeric tether molecule;
(iii) illuminating the sample to generate a signal from the tethered individual second proximity probes; and
(iv) detecting the target analyte by observing the signals from the tethered second proximity probes.

Steps (i) and (ii) in such embodiments may be performed sequentially or simultaneously. Preferably, however, they are performed sequentially. Particularly preferably, steps (i) and (ii) are performed sequentially and no washing step is performed in between steps (i) and (ii).

Steps (iii) and (iv) in such embodiments are performed after steps (i) and (ii). Step (iv) is performed after step (iii). No washing step is required between steps (ii) and step (iii), but a washing step can be performed if desired.

In some embodiments of the invention the second proximity probe is bound to a reporter enzyme, nanoparticle or fluorescent probe. The binding may be via a covalent interaction or via a non-covalent interaction such as those discussed above in the context of the tether molecule or in the context of the first proximity probe. Thus, the second proximity probe may (independently of the nature of the first proximity probe) comprise or consist of a reporter-enzyme-conjugated antibody, lectin, soluble cell surface receptor, combinatorially derived protein from phage display or ribosome display, carbohydrate, aptamer, affimer, affibody, affilin, affitin, alphabody, anticalin, avimer, DARPin, monobody, enzyme, or oligonucleotide. Preferably the second proximity probe comprises or consists of an antibody which is conjugated to a reporter enzyme or a fluorophore.

The second proximity probe may be bound to a plurality of reporter enzymes, nanoparticles or fluorescent probes such that each probe molecule is multiply labeled. The multiple labels increase the signal, whilst still allowing localisation of the signals to a single probe molecule.

The second proximity probe may already be conjugated to the reporter enzyme or a fluorophore prior to contacting the sample. Alternatively, the second proximity probe may contact the sample prior to conjugation of the reporter enzyme and the reporter enzyme may be conjugated to the second proximity probe simultaneously with, or after, contacting the sample.

Thus, in some embodiments the invention provides a method for determining the presence of a target analyte in a liquid sample, comprising the steps of:
(i) contacting and incubating the sample with a first proximity probe comprising an analyte-binding domain having specificity for the target analyte, wherein the first proximity probe is tethered to a solid support by a polymeric or biopolymeric tether molecule;
(ii) contacting and incubating the sample with a second proximity probe comprising an analyte-binding domain having specificity for the target analyte, wherein the second proximity probe is conjugated to a reporter enzyme or fluorophore prior to contacting the sample;
(iii) adding a fluorogenic substrate of the reporter enzyme to the sample to generate a fluorescent reaction product;
(iv) illuminating the sample to cause the fluorescent reaction product to fluoresce; and
(v) detecting the motion of the tethered target analyte by observing fluorescence from the fluorescent reaction product or fluorophore.

Steps (i) and (ii) in such embodiments may be performed sequentially or simultaneously. Preferably, however, they are performed sequentially. Optionally, steps (i) and (ii) are performed sequentially and a washing step is performed in between steps (i) and (ii). Any suitable washing buffer may be employed for the washing step. The washing step helps to remove molecules from the sample which have not been bound by the first proximity probe or non-specifically bound to the support, thereby helping to minimise any background fluorescence.

Steps (iii), (iv) and (v) in such embodiments are performed after steps (i) and (ii). Preferably a further washing step is performed between steps (ii) and (iii) in order to remove any unbound molecules of the second proximity probe. Steps (iv) and (v) are performed after step (iii). Steps (iv) and (v) are performed simultaneously with each other.

In some embodiments the invention provides a method for determining the presence of a target analyte in a liquid sample, comprising the steps of:
(i) contacting and incubating the sample with a first proximity probe comprising an analyte-binding domain having specificity for the target analyte, wherein the first proximity probe is tethered to a solid support by a polymeric or biopolymeric tether molecule;
(ii) contacting and incubating the sample with a second proximity probe comprising an analyte-binding domain having specificity for the target analyte, wherein the second proximity probe is conjugated to a reporter enzyme simultaneously with or after contacting the sample;
(iii) adding a fluorogenic substrate of the reporter enzyme to the sample to generate a fluorescent reaction product;
(iv) illuminating the sample to cause the fluorescent reaction product to fluoresce; and
(v) detecting the motion of the tethered target analyte by observing fluorescence from the fluorescent reaction product or fluorophore.

Steps (i) and (ii) in such embodiments may be performed sequentially or simultaneously. Preferably, however, they are performed sequentially. Particularly preferably, steps (i) and (ii) are performed sequentially and a washing step is performed in between steps (i) and (ii). Any suitable washing buffer may be employed for the washing step. The washing step helps to remove molecules from the sample which have not been bound by the first proximity probe or non-specifically bound to the support, thereby helping to minimise any background fluorescence.

Steps (iii), (iv) and (v) in such embodiments are performed after steps (i) and (ii). Preferably a further washing step is performed between steps (ii) and (iii) in order to remove any unbound molecules of the second proximity probe. Steps (iv) and (v) are performed after step (iii). Steps (iv) and (v) are performed simultaneously with each other.

In all embodiments of the invention where the second proximity probe is conjugated to a reporter enzyme, the reporter enzyme is an enzyme which can act on a suitable substrate to generate a fluorescent reaction product and thereby provide a signal indicating the presence of analyte bound to the second proximity probe. Suitable enzymes include, but are not limited to, alkaline phosphatase (AP), horseradish peroxidase (HRP), and beta galactosidase. Suitable fluorogenic substrates include, but are not limited to, resorufin-β-d-galactopyranoside (RGP), (10-Acetyl-3,7-Dihydroxyphenoxazine (ADHP), 4-Methylumbelliferyl Phosphate (MUP), Fluorescein Di Phosphate (FDP), or QuantaBlu™ or QuantaRed™ available from ThermoFisher Scientific. The skilled person will readily be able to select appropriate reporter enzymes and substrates for any given application of the invention.

In further embodiments of the invention, the second proximity probe comprises an oligonucleotide sequence for fluorescence in-situ hybridisation (FISH) or DNA-PAINT probes (reversibly binding fluorescently labelled oligonucleotides). In such embodiments, the second proximity probe may consist of such an oligonucleotide sequence or the second proximity probe may comprise the oligonucleotide sequence which is suitable for FISH together with other components.

In embodiments of the invention where the second proximity probe comprises an oligonucleotide for FISH, the second proximity probe must then be hybridised with a FISH probe which comprises a fluorophore and an oligonucleotide sequence complementary to the oligonucleotide sequence of the second proximity probe.

Thus, in some embodiments the invention provides a method for determining the presence of a target analyte in a liquid sample, comprising the steps of:
(i) contacting and incubating the sample with a first proximity probe comprising an analyte-binding domain having specificity for the target analyte, wherein the first proximity probe is tethered to a solid support by a polymeric or biopolymeric tether molecule;
(ii) contacting and incubating the sample with a second proximity probe comprising an analyte-binding domain having specificity for the target analyte, wherein the second proximity probe comprises an oligonucleotide sequence for fluorescence in-situ hybridisation (FISH) or DNA-PAINT;
(iii) hybridising the second proximity probe with a FISH probe which comprises a fluorophore and an oligonucleotide sequence complementary to the oligonucleotide sequence of the second proximity probe;
(iv) illuminating the sample to cause the fluorophore to fluoresce; and
(v) detecting the motion of the tethered target analyte by observing fluorescence from the fluorophore.

Steps (i) and (ii) in such embodiments may be performed sequentially or simultaneously. Preferably, however, they are performed sequentially (particularly preferably with step (ii) following step (i)). Optionally, steps (i) and (ii) are performed sequentially and a washing step is performed in between steps (i) and (ii). Any suitable washing buffer may be employed for the washing step. The washing step helps to remove molecules from the sample which have not been bound by the first proximity probe or non-specifically bound to the support, thereby helping to minimise any background fluorescence.

Steps (iii), (iv) and (v) in such embodiments are performed after steps (i) and (ii). Optionally a further washing step is performed between steps (ii) and (iii) in order to remove any unbound molecules of the second proximity probe. Steps (iv) and (v) are performed after step (iii). Steps (iv) and (v) are performed simultaneously with each other.

A plurality of distinct second proximity probes, and therefore a plurality of distinct target analytes, can be distinguished by employing a plurality of second proximity probes each comprising a different oligonucleotide sequence for FISH such that a plurality of different FISH probes can be employed which comprise oligonucleotide sequences complementary to those of the different second proximity probes. Distinct analytes can then be distinguished by means of each of the plurality of FISH probes carrying distinct fluorophores or if DNA-PAINT is used, with one fluorophophore attached to different oligo probes designed to dynamically bind to a conjugate, a number of analytes can be imaged sequentially (one-colour imaging). Distinct fluorophores may be spectrally separated during detection, yielding information on the identity of the secondary proximity probe and therefore the identity of the target analyte. Alternatively, a plurality of FISH probes may be employed carrying the same fluorophores but having oligonucleotide sequences complementary to different second proximity probes. In such embodiments, the plurality of FISH probes can be introduced sequentially with washing steps in between, so that identification of distinct target analytes happens sequentially. The sequential approach reduces demands on the complexity of the detection device.

A plurality of tethers of different lengths can be used to distinguish multiple analytes in the sample, where first proximity probes for different analytes are attached to tethers of different length so that different analytes exhibit different characteristics of motion, such as the range of movement, which are used to identify which analyte is observed. For example as shown below a DNA tether of 2 kB in length can be distinguished from a DNA tether of 3 kB in length.

Detecting multiple analytes in a sample can be achieved by spatially separating different species of tethered first proximity probes so that different areas of the solid support are known to have different first proximity probes attached.

Detecting multiple analytes in a sample can be achieved by using reporter probes specific to each analyte. Each reporter probes can be labelled differently. For example, different reporter probes can carry different fluorophores. If the signal is detected by iSCAT, different reporter probes can carry nanoparticles with different mass.

The first and second proximity probes in all embodiments of the invention should have affinity for different binding sites on the target analyte. The binding sites should be spatially distinct. In one embodiment the first and second proximity probes may have affinity for different epitopes on an antigen. In another embodiment the first and second proximity probes may have affinity for the same epitope on a multimeric antigen.

The solid support should be (at least partially) transparent to light in the region 400 nm to 1000 nm or sub-regions thereof. Preferably the solid support should be at least partially transparent to light of at least one of the following wavelengths: 405 nm, 473 nm, 488 nm, 532 nm, 561 nm, 638 nm, 640 nm, 710 nm and/or 850 nm. The solid support should preferably have a planar surface or a surface of which at least a portion is planar and to which planar surface or planar portion the tether molecule is bound. The solid support may be a coverslip, glass microscope slide, optical fiber, or prism. Alternatively, the solid support may be a surface of a microtiter plate (e.g. a 6, 24, 96, 384 or 1536-well microtiter plate) or at least a portion thereof, preferably the bottom of a well thereof. In another alternative the solid support may be a surface of a microfluidic chamber (in a microfluidic chip) or at least a portion thereof. Preferably the solid support comprises, consists essentially of or consists of glass or an optically transparent polymer.

Following the incubation of the second proximity probe (and following the conjugation of the reporter enzyme (if necessary), where this step is carried out after incubation) a washing step is preferably performed using a buffer solution. Preferably, the buffer solution comprises a mixture of BSA and a detergent such as Triton X-100 in PBS (phosphate-buffered saline). Other standard ELISA wash buffers known to those skilled in the art may also be used. The wash is intended to remove any unbound second proximity probe and/or non-conjugated reporter enzyme.

Detection of the analyte is performed by detection of fluorescence arising from the fluorophore or fluorescent reaction product generated by the interaction of the fluorogenic substrate with the reporter enzyme or using FISH probes. The method of the invention is therefore essentially based in principle on a conventional “sandwich”-type ELISA immunoassay but, uniquely, the first proximity probe is tethered to the solid support so that although the probe is affixed to the support, the first proximity probe can move in a limited range.

Even where a washing step is performed to remove unbound reporter probes and/or reporter enzymes, there will inevitably be an unavoidable degree of non-specific binding of analytes, reporter enzymes, and/or second proximity probes to the solid support. Such non-specifically bound reporter enzymes and/or second proximity probes will therefore generate fluorescence, thereby giving rise to a background signal and/or “false positive” detection of analyte. Such non-specifically bound species therefore limit the accuracy of conventional assays, particularly when the analyte is present in very low concentrations. The present inventors have surprisingly found that, by tethering the first proximity probe to the support such that the proximity probes and the analyte can move in a limited range, it is possible to distinguish between such background or “false positive” signals on the one hand and, on the other hand, genuine signals arising from second proximity probes which are specifically bound to analytes captured by the tethered first proximity probe.

The fluorescence referred to herein is the detection of single, or a small number of, fluorescent molecules, i.e. the photons emitted by an individual fluorophore, or a few fluorophores attached to individual (single) tethers, rather than emission from a bulk population, i.e., the photons emitted by a fluorescent solution or surface. Where a second proximity probe is non-specifically bound to the solid support, or bound to an analyte which is itself non-specifically bound to the solid support, the probe will not move, and therefore any fluorescence generated by the second proximity probe or by reporter molecules generated by interaction of the reporter enzyme with the substrate will be highly localised. If the standard deviation of the detected positions of non-specifically bound molecules is calculated, the value may typically be of the order of 50 nm. In contrast, molecules of the target analyte which are specifically bound to the tethered first proximity probe will have a greater range of movement due to the flexibility of the tether and its ability to undergo stochastic (Brownian) or controlled motion in solution. Optionally controlled movement of tethered molecules can be achieved by attaching magnetic materials to the tethered molecule(s) and subjecting them to a changing electro-magnetic field or changing velocity (speed and direction) of the fluid.

Molecules of the second proximity probe which are specifically bound to such tethered analyte molecules will therefore also undergo distinct and detectable movement within a limited range. As a result, when such second proximity probes scatter or fluoresce and/or generate fluorescent reporter molecules, a variety of measurements can be made to distinguish them from non-specifically immobilized second proximity probes and from non-immobilised second proximity probes. The distinction can be achieved by taking single-frame long-exposure images, in which a tethered probe appear to smear over a limited range, and a non-specifically immobilised probe appear to be a small point, and a non-immobilised probes is practically invisible because its unlimited range of movement. The distinction can be also achieved by taking consecutive short-exposure images, in which a tethered probe exhibits a limited range of movement, and a non-specifically immobilised probe exhibits no movement beyond the uncertainty of the experiment, and a non-immobilised probe exhibits practically unlimited range of movement and is usually only observed briefly and diffuses away from the volume of solution being imaged.

In a preferred embodiment, detecting the target analyte therefore comprises monitoring the spatial distribution of photons emitted by fluorophores in the second proximity probe or by fluorescent reaction products arising from the reaction between the substrate and the reporter enzyme or using FISH or DNA-PAINT probes.

In an alternative embodiment the level of light scatter is measured and the motion of the target analyte followed over time.

In some embodiments of the invention, the first proximity probe may itself be conjugated to a fluorophore. In such embodiments, fluorescence will therefore arise not only from the fluorescent species associated with the second proximity probe but also from the fluorophore conjugated to the first proximity probe. The colocalisation of the fluorescence from both first and second proximity probes can be employed in order to further improve the specificity of detection of specifically-bound target analyte molecules.

In some embodiments of the invention, the first proximity probe may itself be conjugated to a fluorophore which acts a fluorescence energy transfer (FRET) donor in one fluorescence channel (channel 1). In such embodiments, fluorescence will therefore arise not only from the fluorescent species associated with the second proximity probe (FRET acceptor, fluorescence in channel 2) but also from the fluorophore conjugated to the first proximity probe. The presence of fluorescence emission from the second proximity probes in channel 2 that are excited by channel 1 excitation can be employed to measure the presence of both the first proximity probe and the second proximity probe in one complex. In order to further improve the specificity of detection of specifically-bound target analyte molecules, the absence of FRET emission will indicate non-specifically bound second proximity probe complexes.

The spatial distribution of fluorescence photons is monitored in the plane of the solid support. The solid support (or the planar portion thereof) may be said to define an xy plane, and therefore the spatial distribution of photons may be monitored and plotted in an xy coordinate system.

In order to cause fluorescence which can be employed to detect the target analyte, the sample is illuminated. This causes the fluorophore or fluorescent reaction product to fluoresce provided that the illumination is at a wavelength capable of exciting fluorescence.

It is particularly preferable that illumination is performed by means of total internal reflection fluorescence microscopy. The use of total internal reflection (TIR) fluorescence microscopy as a means of detection allows for a further increase in the accuracy of the method of the present invention, enhancing the ability of the methods of the invention to detect extremely low concentrations of target analyte and to distinguish between specific and nonspecific binding. TIR makes use of a phenomenon known as the “evanescent wave” or “evanescent field” and this phenomenon can be exploited in the methods of the present invention to further improve on methods known in the art.

Where an incident light beam undergoes TIR at the boundary between two media having different refractive indices, an evanescent illumination field is generated which extends several hundred nanometres into the medium with the lower refractive index. The penetration depth d is dependent on the wavelength λ(i) of the incident illumination, the angle of incidence θ and the refractive indices n₁ and n₂ of the media:

d=λ(i)/4π×(n₁² sin²θ−n₂²)^{-1/2}

The strength of the evanescent field decays exponentially as a function of distance from the interface:

E(z)=E(0)exp(−z/d)

where E(z) is the energy at a perpendicular distance z from the interface, and E(0) is the energy at the interface.

Close to the interface, typically within the first 150 nm, this field is capable of exciting fluorophores. At a sufficiently high power density of illumination, a fluorophore can undergo rapid photobleaching. The rate of photobleaching will be faster in regions where the evanescent field strength is greater, i.e. close to the interface, than in regions where the evanescent field strength is weaker, i.e. further from the interface. The intensity and photobleaching rates may typically be up to 4× higher for fluorophores within the first 50 nm of to the interface compared to molecules which are further away than 150 nm from the interface.

In the methods of the present invention this phenomenon can therefore be exploited to further refine the detection of specifically and non-specifically bound species. As described above, some molecules of target analyte, second proximity probe, and/or reporter enzymes will inevitably bind non-specifically to the solid support. At the surface of the solid support, these non-specifically bound molecules are close to the interface between the solid support and the sample and are therefore in the region where the evanescent field is strongest. Fluorescent species in this region will therefore emit more photons, undergo photobleaching more rapidly, exhibit a shorter diffusion length after becoming fluorescent, and optionally generate a different image if PSF (point spread function) engineering is employed, than fluorescent species further away from the interface. Due to the presence of the tether molecule, specifically bound target analyte molecules (i.e. those which are bound to the tethered first proximity probe) will be located, on average, further away from the interface, where the evanescent field is weaker. When molecules of the second proximity probe interact with such specifically-bound target analyte molecules, these will also be located in this region where the evanescent field is weaker. Consequently, the photon emission and photobleaching rates arising from the fluorophores and/or fluorescent reaction products are detectably lower than those arising closer to the interface. Such fluorescing species will also undergo longer diffusion, and generate a different image under PSF modifications.

PSF modifications can be any aberrations introduced into the optical path of the microscope used for this assay which enhance the ability to measure the position of fluorophore in the direction perpendicular to the solid support. An efficient means of introducing one such aberration called astigmatism is to place a weak cylindrical lens (for example with a focal length of 2 metres) close to the camera which the fluorophores are imaged on. The cylindrical lens causes an asymmetry of the PSF which depends on the z-position of the fluorophore around a few hundred nanometres of the focal plane of a high numerical aperture objective. If the focal plane coincides approximately with the liquid/solid interface of the sample, fluorophores which originate from or are bound on tethers have a different image than fluorophores close to the interface. The images resemble ellipses orientated along the x-axis and y-axis respectively. These different images can be quantified by estimating the width of the ellipse in x and y dimensions. These numerical values of these widths along the x and y axes can be used to calculate the position of the fluorophore with respect to the focal plane, which allows non-specifically bound molecules and specifically bound molecules to be further distinguished.

Preferably the light source employed for illumination is a laser, particularly preferably a laser which provides a power density of from about 5 kW/cm² to about 10 kW/cm² at an excitation wavelength (i.e. a wavelength where the fluorescent species has a significant absorption cross section). Common laser wavelengths that may be employed include, but are not limited to, 405 nm, 473 nm, 488 nm, 532 nm, 561 nm, 638 nm, 640 nm and/or 850 nm. The skilled person will readily be able to select an appropriate laser power and wavelength, having regard to the nature of the fluorescent species which is employed for detection of the target analyte. The use of lasers for illumination is particularly preferable in order to generate TIR and an evanescent field.

Taken together, these phenomena allow us to distinguish spatiotemporal signatures from fluorophores generated from specifically bound complexes versus fluorophores associated with non-specifically bound complexes.

In a particularly preferred embodiment, detecting the analyte therefore comprises monitoring the spatiotemporal distribution of photons emitted by fluorescing species.

The time resolution required for monitoring should either be sufficiently high (preferably 10 ms or faster) to detect individual fluorophore generation, diffusion and photobleaching events, or sufficiently slow (preferably 100 ms or slower) to average several such events. In the latter case, images corresponding to specifically bound analytes would appear broader than images from non-specifically bound analytes due to a longer diffusion period of the reporter molecule as well as dynamics of the tether molecule.

Due to the high spatiotemporal resolution of the methods of the invention, and the ability to discriminate between specific and non-specific binding, the methods of the present invention are capable of detecting the presence of target analytes down to the level of individual molecules (or other relevant entities, such complexes of molecules, clusters, aggregates, viruses etc)

In some embodiments the method of the present invention may be employed for parallel detection of a plurality (e.g. at least two, at least three, or more) of target analytes. Detection of a plurality of analytes will typically require the use of a plurality of first (“capture”) proximity probes and a plurality of second (“reporter”) proximity probes having specificity for different analytes.

In such embodiments the present invention may therefore provide a method for determining the presence of a plurality of target analytes in a liquid sample, comprising the steps of:
(i) contacting and incubating the sample with a plurality of first proximity probes, each first proximity probe comprising an analyte-binding domain having specificity for one of the plurality of target analytes, wherein each of the first proximity probes is tethered to a solid support by a polymeric or biopolymeric tether molecule;
(ii) contacting and incubating the sample with a plurality of second proximity probes, each second proximity probe comprising an analyte-binding domain having specificity for one of the plurality of target analytes, wherein each of the plurality of second proximity probes independently of one another
   a. comprises a fluorophore; or
   b. is conjugated to a reporter enzyme prior to contacting the sample; or
   c. is conjugated to a reporter enzyme simultaneously with or after contacting the sample; or
   d. comprises an oligonucleotide sequence for fluorescence in-situ hybridisation (FISH) or DNA-PAINT; or
   e. comprising nanoparticle of different size
(iii) a. if any of the plurality of second proximity probes is conjugated to a reporter enzyme, adding a fluorogenic substrate of the reporter enzyme to the sample to generate a fluorescent reaction product; or
   b. if any of the plurality of second proximity probes comprises an oligonucleotide sequence for fluorescence in-situ hybridisation (FISH), hybridising the second proximity probe with a FISH probe which comprises a fluorophore and an oligonucleotide sequence complementary to the oligonucleotide sequence of the second proximity probe;
   c. if any of the plurality of second proximity probes comprises different size of nanoparticles for plurality of analytes;
(iv) illuminating the sample to cause the fluorophores and/or fluorescent reaction products to fluoresce; or light scattering of nanoparticles; and
(v) detecting the plurality of target analytes by observing fluorescence from the fluorophores and/or fluorescent reaction products or plurality of scattering by nanoparticle iSCAT

In such embodiments there are self-evidently more than two proximity probes in total. Thus the terms “first” and “second” in such embodiments should not be taken literally and may rather be taken as functional descriptions which extend the general concept of the invention, wherein the “first” proximity probes each act as “capture” probes which bind a target analyte to the support via the tether, and the “second” proximity probes act as “reporter” probes which enable the detection of the bound target analyte.

Collectively, the plurality of first proximity probes should have specificity for all of the plurality of target analytes, with each of the first proximity probes having specificity for a different one of the plurality of target analytes. Thus, if there are two target analytes, there should be two first proximity probes, each proximity probe having specificity for a different one of the two analytes; if there are three target analytes, there should be three first proximity probes, each proximity probe having specificity for a different one of the three analytes; if there are four target analytes, there should be four first proximity probes, each proximity probe having specificity for a different one of the four analytes; and so on for greater numbers of target analytes.

Similarly, the plurality of second proximity probes should collectively have specificity for all of the plurality of target analytes, with each of the second proximity probes having specificity for a different one of the plurality of target analytes. Thus, if there are two target analytes, there should be two second proximity probes, each proximity probe having specificity for a different one of the two analytes; if there are three target analytes, there should be three second proximity probes, each proximity probe having specificity for a different one of the three analytes; if there are four target analytes, there should be four second proximity probes, each proximity probe having specificity for a different one of the four analytes; and so on for greater numbers of target analytes.

The same considerations as set forth elsewhere herein with regard to the order of the method steps, the nature of the sample and target analytes, the nature of the proximity probes, the nature of the tether, the nature of the support, etc., apply mutatis mutandis to the methods for determining the presence of a plurality of target analytes.

In particular, where a plurality of target analytes are present, these may each independently of one another be a biomarker indicating the presence of a disease such as cancer or a neurodegenerative disease, or indicating the presence of an infection. A typical biomarker may for example be a hormone, a peptide (e.g. a protein or enzyme), carbohydrate, antibody, or nucleic acid. Typically each of the plurality of target analytes may be antigens with the proviso that they are different to one another.

Each of the first proximity probes may independently of one another comprise or consist of an antibody, lectin, soluble cell surface receptor, combinatorially derived protein from phage display or ribosome display, carbohydrate, aptamer, affimer, affibody, affilin, affitin, alphabody, anticalin, avimer, DARPin, monobody, or combinations thereof, with the proviso that each of the first proximity probes should have specificity for a different target analyte. Preferably each of the first proximity probes is an antibody.

Each of the second proximity probes may (independently of the nature of the first proximity probes and independently of one another) comprise or consist of an antibody as defined above, a lectin, soluble cell surface receptor, combinatorially derived protein from phage display or ribosome display, carbohydrate, aptamer, affimer, affibody, affilin, affitin, alphabody, anticalin, avimer, DARPin, monobody, enzyme, or oligonucleotide, with the proviso that each of the second proximity probes should have specificity for a different target analyte.

Each of the second proximity probes may independently of one another comprise a fluorophore or be conjugated to a reporter enzyme or comprise an oligonucleotide sequence for fluorescence in-situ hybridisation (FISH). Where a plurality of second proximity probes each comprise a fluorophore the fluorophores may be the same or different. Where a plurality of second proximity probes are each conjugated to a reporter enzyme the enzyme may be the same or different. Thus, a plurality of second proximity probes may each independently of one another be conjugated to alkaline phosphatase (AP), horseradish peroxidase (HRP), or beta galactosidase, for example. Where a plurality of second proximity probes are conjugated to reporter enzymes, the fluorogenic substrates for each of the second proximity probes may be the same or different. Each of the plurality of second proximity probes may independently be conjugated to its respective reporter enzyme prior to contacting the sample. Alternatively, and independently of one another, each of the plurality of second proximity probes may contact the sample prior to conjugation of its respective reporter enzyme and the reporter enzyme may be conjugated to the probe simultaneously with, or after, contacting the sample. Where a plurality of second proximity probes each comprise an oligonucleotide sequence for FISH the oligonucleotide sequences may be the same or different and the fluorophores in the complementary FISH probes may be the same or different as described elsewhere herein.

Each of the plurality of first proximity probes is tethered to a solid support by a polymeric or biopolymeric tether molecule. In one embodiment each of the plurality of first proximity probes is tethered to the support by a separate tether molecule such that each tether molecule carries one type of first proximity probe. Preferably, however, each of the plurality of first proximity probes is conjugated to the same tether molecule. Each of the plurality of first proximity probes in such embodiments is tethered to a different site on the same tether molecule. dsDNA and DNA origami structures are particularly suitable tether molecules for such purposes. DNA origami structures may have a plurality of binding sites for proximity probes and thus a plurality of first proximity probes (for example two, three, four or more first proximity probes) having specificity for different antigens may be conjugated to the origami structure. Optionally, multiple DNA origami structures may be employed simultaneously in this fashion in order to allow further discrimination between different origamis and/or different capture probe sites and thus further increase the number of analytes which can be detected in parallel. Where multiple DNA origami structures are employed, the first proximity probes may first be conjugated to the origami structures before the origami structures are tethered to the solid support.

In a further aspect the present invention provides a solid support as herein described, preferably a coverslip, glass microscope slide, optical fiber, prism, microtiter plate, or microfluidic chip, having a first proximity probe tethered thereto by a polymeric or biopolymeric tether molecule. All discussion herein regarding the nature of the solid support, the nature of the first proximity probe, and the nature of the tether molecule, including the means of covalent or noncovalent attachment of the tether molecule to the support and of covalent or noncovalent attachment of the proximity probe to the tether molecule, applies mutatis mutandis to this aspect of the invention. Such supports should self-evidently be suitable for use in the methods of the invention.

The present invention will now be illustrated further by the following non-limiting Examples and the Figures, in which:

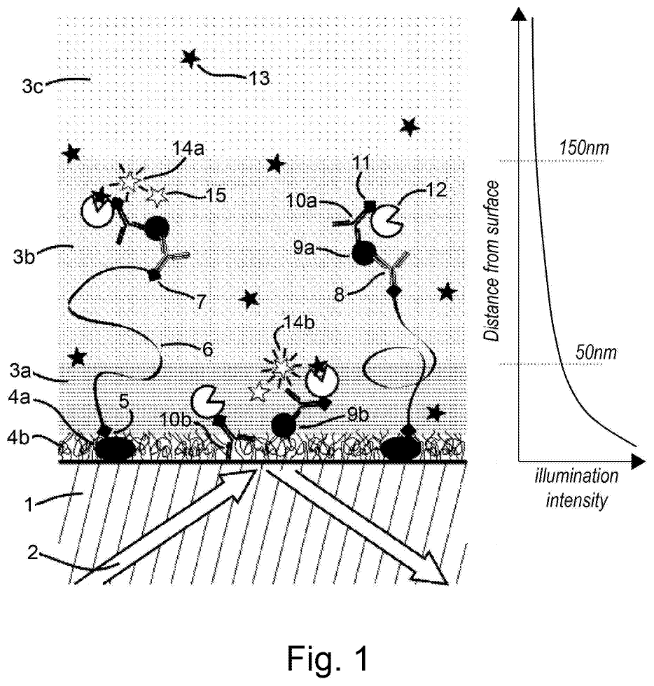

FIG. 1 shows a schematic illustration of one embodiment of the invention employing TIR fluorescence detection, as described in Example 1.

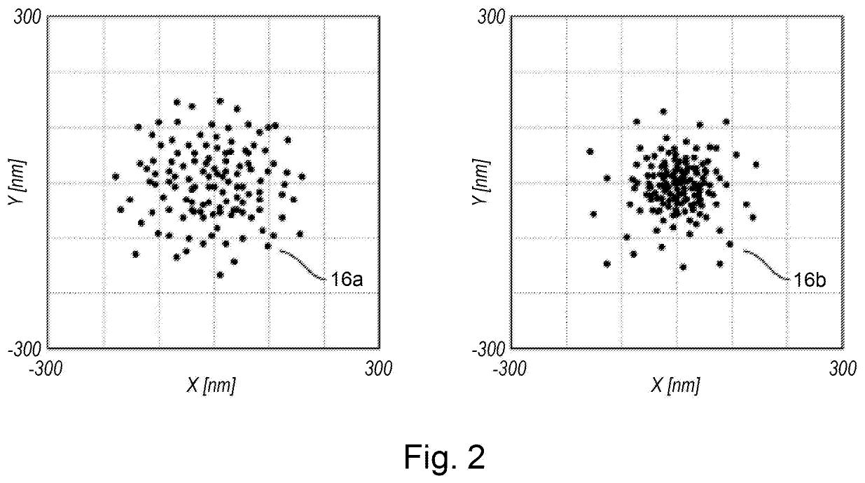

FIG. 2 shows an illustration of spatial distributions of photons indicating specific (left) and non-specific (right) binding of analytes and reporter probes.

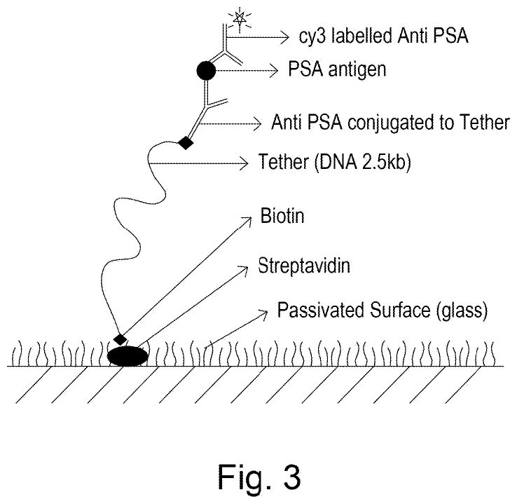

FIG. 3 illustrates a schematic illustration of one embodiment of the invention employing fluorescence detection. The assay uses a passivated glass surface, to which streptavidin molecules are attached. The tether is a DNA molecule of around 2.5 kb attached via biotin. The DNA tether carries an anti-PSA conjugated thereto. The probe detected is PSA antigen, which is affinity labelled with a secondary Cy3 labelled anti PSA.

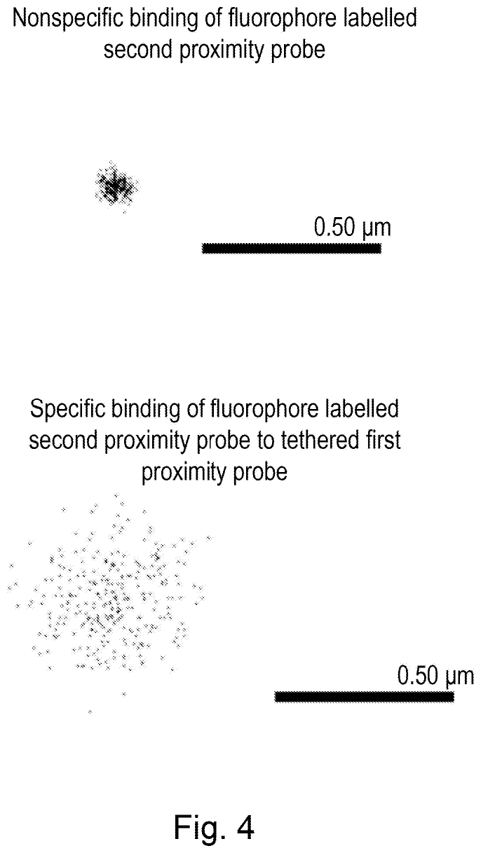

FIG. 4 shows an illustration of spatial distributions of photons indicating non-specific (upper) and specific (lower) binding of analytes and reporter probes with a scale bar of 0.5 micrometers. The image of PSA captured by anti-PSA tethered DNA and detected with Cy3B conjugated anti-PSA. The sample was illuminated with 532 nm (green) laser using TIRF microscopy and the imaged using 10 ms time resolution.

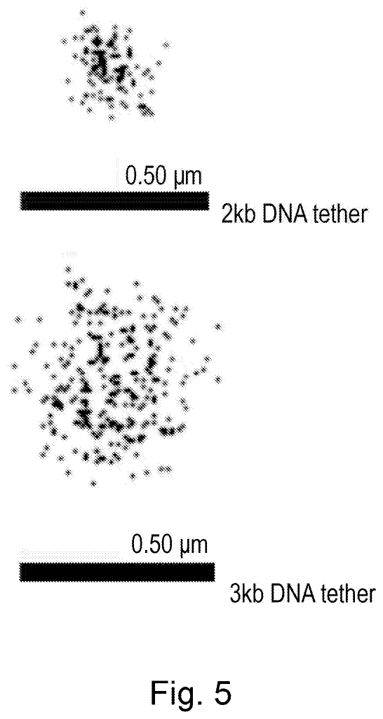

FIG. 5 shows an illustration of spatial distributions of photons indicating specific binding on 2 kb tether (upper) and specific binding on 3 kb tether (lower) of analytes and reporter probes with a scale bar of 0.5 micrometers. The small molecule digoxigenin tethered on the 2 kb and 3 kb DNA bound by anti-digoxigenin is detected by Cy3 conjugated anti-mouse.

### EXAMPLE 1

FIG. 1 shows an illustrative embodiment of a method according to the invention. In this embodiment, an assay is performed on the surface of a glass support (1). A total internal reflection (TIR) illumination scheme (2) is employed. TIR generates an evanescent illumination field that decays exponentially within a few hundred nanometres of the interface (illustrated on the right of the Figure). The medium can be conceptually divided into regions of high (3a), intermediate (3b) and low (3c) illumination density. The surface of (1) is silanized and passivated using biotinylated BSA (4a) and Tween-20 (4b). Interaction (5), which can, for instance, be a covalent bond or a biotin-streptavidin-biotin interaction, leads to the binding of a 1 kb dsDNA tether (6) to the surface (1) via BSA (4a). Interaction (7), which can, for instance, be a biotin-streptavidin-biotin interaction, binds capture antibodies (8) onto the dsDNA tether.

A serum sample was loaded onto the functionalized surface (1) and incubated. After incubation, antigen (9a) is specifically bound by capture antibody (8). Some antigens (9b) will also bind non-specifically to surface (1). Reporter antibodies (10a) are added and incubated and they bind specifically to antigens (9a), (9b). Some reporter antibodies (10b) will also bind non-specifically to the surface (1).

A reporter enzyme, β-galactosidase (12) is added and binds to the reporter antibody (10a), (10b), here via a biotin-streptavidin interaction (11). Fluorogenic substrate resorufin-β-d-galactopyranoside (RGP) (13) is added and converted by enzyme (12) to fluorescent reporter molecules (14a), (14b). Some reporter enzymes will also non-specifically bind to the surface (1) (this is not shown in FIG. 1).

Laser illumination (2) with high power density forces the nascent fluorophores (14a), (14b) to emit several thousand photons before becoming photobleached (15). Fluorophores (14b) in region (3a) emit more photons, photobleach faster, and have less time to diffuse away from the location of the reporter enzyme. Fluorophores (14a) in region (3b) emit fewer photons, survive longer and are able to diffuse further away from the location of the reporter enzyme. The photons are collected via an optical system and imaged onto a camera. Fluorophores which diffuse into region (3c) and the bulk medium are illuminated only weakly and are not detected. Position information contained in photons collected during each camera frame allows the mean position of the fluorophore emitting these photons to be determined within 10 nanometre precision. These mean positions, called localizations, can be plotted in an xy coordinate system as shown in FIG. 2. Each reporter enzyme generates one cluster of localizations. Localizations (16a) from fluorophores (14a) are generated by enzymes on a tethered immunocomplex. These are more disperse compared to localizations (16b) from fluorophores (14b) generated by enzymes associated with non-specifically bound antigens (9b), antibodies (10b) and reporter enzymes. Dynamics of the tether (6) additionally result in motion of the enzyme-linked immunocomplex bound on the tether, resulting in disperse locations of fluorophore generation recorded as a function of time. The spatiotemporal distribution of localizations, and the number of photons collected from individual fluorophores which are being generated continuously, yield unique signatures for the presence of tethered, enzyme-linked, intact immunocomplexes. Localizations originating from fluorophores in regions (3a) and (3b) can be further distinguished by implementing engineered astigmatic or double helix point spread functions which have a strong dependence on the height of the fluorophore with respect to the glass surface.

### EXAMPLE 2

This example illustrates a simple protocol for the passivation of a microscope slide or coverslip. This protocol may also be employed for treating microfluidic chips.

The coverslip is placed in a staining jar and rinsed with ultrapure water. The staining jar is then filled with fresh high-purity acetone in order to remove any organic compounds which might otherwise interfere with fluorescence measurements. The staining jar containing the coverslip and acetone is placed in a sonicator and sonicated for about 20 minutes. Following sonication, the acetone is discarded and the coverslip rinsed again with ultrapure water. After rinsing, the staining jar is filled with 1M KOH and sonicated again for about 20 minutes. The KOH is discarded and the coverslip is rinsed again with ultrapure water. A further sonication step of about 20 minutes is performed with the coverslip immersed in ultrapure water and then the coverslip is dried using nitrogen gas. Optionally, the coverslip may be further subjected to etching using piranha solution (a mixture of H₂SO₄ and H₂O₂) or by plasma treatment in order to improve the surface quality and to assist in obtaining high-quality surface passivation in the subsequent steps.

Passivation to prevent non-specific binding of biomarkers can be performed using naturally occurring proteins such as bovine serum albumin (BSA) or synthetic compounds such as polyethylene glycol (PEG). The following steps describe the use of BSA and PEG as passivating agents but the skilled person will be able to adapt these as required for alternative passivating agents. After the coverslip has been cleaned as described above, and optionally further treated with piranha solution or plasma treatment, the coverslip is treated with APTES (3-aminopropyl triethoxy silane), APTMS (3-aminopropyl trimethoxysilane) or dichlorodimethylsilane (DDS)-Tween-20.

To passivate coverslips which have been etched with KOH and/or treated with DDS-Tween-20, 100 μl of aqueous solution are added which contain a saturating concentration of BSA mixed with 0.2 mg/ml biotinylated BSA.

To passivate coverslips with PEG, 100 μl of an aqueous mixture are added which contain at least 0.2 mg biotinylated NHS-ester PEG and at least 8 mg of NHS-ester mPEG in 0.1 M of fresh sodium bicarbonate buffer (pH 8.5).

A sandwich of coverslips is made which is immersed in the passivating solution and these are left in a dark, moist chamber undisturbed for at least 3 hours (overnight incubation is recommended). Afterwards, the coverslips are separated and rinsed with ultrapure water before being dried with nitrogen gas.

### EXAMPLE 3

This Example illustrates one possible implementation of the method of the invention.

dsDNA is employed as a tether molecule in this Example, although this protocol can be adapted for use with any suitable tether. A passivated microfluidic chamber is employed as the solid support, although this protocol can also be adapted for any form of solid support contemplated herein. The microfluidic chamber may be passivated according to the protocol described in Example 2, for instance.

The concentration of dsDNA solution is optimised to allow about 300 to 400 tether molecules to be immobilised on the solid support. The tether molecule solution is introduced into the microfluidic chamber and incubated for about 2 minutes. Unbound tether molecules are removed by washing with an appropriate buffer and then 50 μl of a 10 nM solution of the first proximity probe are introduced into the microfluidic chamber. This solution is incubated for about 5 minutes and then any unbound probe is removed by washing with at least 100 μl of an appropriate washing buffer.

A sample for analysis is introduced into the microfluidic chamber, such as a drop of blood from a patient. This sample is incubated for about 5 minutes so that any target analytes such as biomarkers in the sample are captured by the tethered first proximity probe.

The chamber is then washed with a wash buffer to remove any unbound molecules, before introducing 50 μl of a 10 nM solution of the second proximity probe which has been conjugated to a reporter enzyme such as Horseradish peroxidase, Alkaline phosphatase or beta galactosidase.

Any unbound molecules of the second proximity probe are removed by flushing the microfluidic chamber with 100 μl of the wash buffer. A fluorogenic substrate for the reporter enzyme is then introduced into the microfluidic chamber in order to allow the reporter enzyme to react with this substrate and produce a fluorescent species.

The microfluidic chamber is then introduced into a TIRF microscope and illuminated with a laser in order to cause the fluorescent reaction product to fluoresce. Fluorophores undergo photobleaching and motion as described elsewhere herein, allowing a low background fluorescence to be maintained while specifically-bound analyte molecules can be detected and distinguished from non-specifically-bound species due to their characteristic spatiotemporal behaviour.

### EXAMPLE 4

This Example illustrates the preparation of a tether having multiple first proximity probes bound thereto, in order to enable parallel detection of multiple analytes.

A DNA origami structure is employed as the tether. A solid support is passivated as described in Example 2 and the origami structure is bound to the support analogously to the dsDNA as described in Example 3.

The DNA origami structure is conjugated to biotin, digoxigenin (DIG) and fluorescein isothiocyanate (FITC). The conjugated biotin provides a first set of binding sites, the conjugated DIG provides a second set of binding sites and the conjugated FITC provides a third set of binding sites. These can be exploited to provide binding sites for three different types of antibodies which act as capture probes.

A first antibody, having specificity for a first antigen, is conjugated to the biotin binding sites via a biotin-strep interaction. A second antibody, having specificity for a second antigen, is conjugated to the DIG binding sites via an Ab-anti-dig interaction. A third antibody, having specificity for a third antigen, is conjugated to the FITC binding sites via an Ab-anti-FITC interaction.

Each of the first, second and third antibodies conjugated to the DNA origami tether in this manner may then be employed as a first proximity probe according to the methods of the invention. Due to their different specificities, this allows the parallel detection of three different antigens, each antigen being a target analyte in the sense of the present invention. In order to perform parallel detection, different fluorophores are conjugated to each of the second proximity probes which may be antibodies, or different enzymes giving rise to different fluorogenic reaction products are employed for detection, so that the fluorescence associated with the three different analytes can be distinguished.

By analogy with this example, parallel detection of four or more different antigens may also be performed by providing a suitable number of different antibodies as first proximity probes. Optionally, multiple DNA origami structures may be employed as tethers, each DNA origami being conjugated to different antibodies and having different characteristic fluorophores associated therewith in order to allow further discrimination between fluorescence originating from different origamis and/or different capture probe sites. In such cases, the capture antibodies need to be incubated to the origami structures (separately) prior to the immobilization of the origami structures in the microfluidic chamber.

### EXAMPLE 5

This Example illustrates the preparation of a tether having multiple lengths and multiple first proximity probes bound thereto, in order to enable parallel detection of multiple analytes.

A mixture of tether molecules that have been conjugated to different first proximity probe is immobilized onto a surface of a microfluidic chamber. Each of the tether molecules have different lengths to one another.

A sample (blood serum) containing biomarkers of interest is injected into the microfluidic chamber. The biomarkers bind to their respective first proximity probes. A second proximity probe containing an enzyme or a fluorophore is used to detect the presence of each biomarker. Due to the different lengths of the tether molecules associated with each first proximity probe, the PSF associated with the signal generated by the second proximity probe will differ. Accordingly, the differences in PSF can be used to assist in assigning fluorescence signal arising from each individual biomarker. This can be further enhanced if different fluorophores are employed in each first/second proximity probe pairing in order to allow additional discrimination between fluorescence signals from different binding sites.

Data shown in FIG. 5 shows the difference between 2 kb and 3 kb tethers where the DNA tether carries an digoxigenin tethered on the 2 kb and 3 kb DNA. The tethered digoxigenin is bound to anti-digoxigenin, which is labelled using a Cy3 conjugated anti-mouse antibody. The 3 kb tethers move over a larger area of the surface than the 2 kb tethers.

### EXAMPLE 6

This Example illustrates the preparation of a tether in combination with Fluorescent in situ hybridization (FISH) probes for parallel detection of multiple analytes. The tether bearing the first proximity probe is immobilized on the surface of the solid support, which is a surface of a microfluidic chamber. The serum containing the biomarkers is then introduced to the microfluidic chamber.

The biomarker specifically binds to the first proximity probe. The second proximity probe (for example, an aptamer or an antibody) is conjugated with a unique nucleic acid sequence. The unbound second proximity probe is then removed. A FISH probe is then employed to detect the presence of the analyte.

If multiple first proximity probes are employed, then appropriate second proximity probes and FISH probes may be employed for parallel detection of multiple analytes. In such a case, FISH probes which are specific for each analyte may be added and detected sequentially. So, for instance, the FISH probe for the first analyte may be added, detected and removed, followed by the FISH probe for the second analyte, and so on until N FISH probes for N analytes have been detected. Different FISH probes could also be decorated with different dyes to allow simultaneous multi-color imaging.

1. A method for determining the presence of a target analyte in a liquid sample, comprising the steps of:
(i) contacting and incubating the sample with a first proximity probe comprising an analyte-binding domain having specificity for the target analyte, wherein the first proximity probe is tethered to a solid support by a polymeric or biopolymeric tether molecule, thereby tethering the target analyte to the support;
(ii) generating signals from individual molecules which are specific to the tethered target analyte; and
(iii) detecting the tethered target analyte by observing the motion of the signals on the solid support.

2. The method for determining the presence of a target analyte in a liquid sample according to claim 1, wherein the signals are fluorescence.

3. The method for determining the presence of a target analyte in a liquid sample according to claim 2, wherein illumination of the sample and detection of the target analyte are performed using total internal reflection fluorescence microscopy.

4. The method for determining the presence of a target analyte in a liquid sample according to claim 1, wherein the signals are light scatter.

5. The method for determining the presence of a target analyte in a liquid sample according to claim 1, further comprising the steps of:
(iv) contacting and incubating the sample with a second proximity probe comprising an analyte-binding domain having specificity for the target analyte, wherein
a. the second proximity probe comprises a fluorophore; or
b. the second proximity probe is conjugated to a reporter enzyme prior to contacting the sample; or
c. the second proximity probe is conjugated to a reporter enzyme simultaneously with or after contacting the sample; or
d. the second proximity probe comprises an oligonucleotide sequence for fluorescence in-situ hybridization (FISH); or
e. the second proximity probe comprises a nanoparticle which generates measurable signal to be identified;
(v) a. if the second proximity probe is conjugated to a reporter enzyme, adding a fluorogenic substrate of the reporter enzyme to the sample to generate a fluorescent reaction product; or
b. if the second proximity probe comprises an oligonucleotide sequence for fluorescence in-situ hybridisation (FISH), hybridising the second proximity probe with a FISH probe which comprises a fluorophore and an oligonucleotide sequence complementary to the oligonucleotide sequence of the second proximity probe;
(vi) illuminating the sample to cause the fluorophore or fluorescent reaction product to fluoresce or cause the nanoparticle to scatter light; and
(vii) detecting the target analyte by observing the motion of the fluorescence or scattering signal from the fluorophore or fluorescent reaction product or nanoparticle.

6. The method according to claim 1 wherein the first proximity probe comprises an antibody, lectin, soluble cell surface receptor, combinatorially derived protein from phage display or ribosome display, carbohydrate, aptamer, affimer, affibody, affilin, affitin, alphabody, anticalin, avimer, DARPin, oligonucleotide, polynucleotide, monobody, or combinations thereof.

7. The method according to claim 5 wherein the second proximity probe comprises an antibody, a lectin, soluble cell surface receptor, combinatorially derived protein from phage display or ribosome display, carbohydrate, aptamer, affimer, affibody, affilin, affitin, alphabody, anticalin, avimer, DARPin, oligonucleotide, polynucleotide, monobody, or enzyme.

8. The method according to claim 7 wherein the second proximity probe comprises a fluorophore or nanoparticle.

9. The method according to claim 7 wherein the second proximity probe is conjugated to a reporter enzyme prior to contacting the sample.

10. The method according to claim 7 wherein the second proximity probe is conjugated to a reporter enzyme simultaneously with or after contacting the sample.

11. The method according to claim 9 wherein the reporter enzyme is alkaline phosphatase (AP), horseradish peroxidase (HRP), or beta galactosidase.

12. The method according to claim 5 wherein the fluorogenic substrate is resorufin-β-d-galactopyranoside (RGP), (10-Acetyl-3,7-Dihydroxyphenoxazine (ADHP), 4-Methylumbelliferyl Phosphate (MUP), or Fluorescein DiPhosphate (FDP).

13. The method according to claim 5 wherein the second proximity probe comprises an oligonucleotide sequence for fluorescence in-situ hybridisation (FISH).

14. The method according to claim 6 wherein the first proximity probe is conjugated to a fluorophore or nanoparticle.

15. The method according to claim 1 wherein the tether molecule comprises a surfactant, lipid, peptide, protein, oligosaccharide, polysaccharide, oligonucleotide or polynucleotide.

16. The method according to claim 1 wherein the tether molecule comprises DNA or RNA.

17. The method according to claim 1 wherein the tether molecule comprises dsDNA or a DNA origami structure.

18. The method according to claim 1 wherein the tether molecule has a mean end-to-end distance of about 50 nm to about 1000 nm in aqueous solution.

19. The method according to claim 1 wherein the tether molecule comprises DNA or RNA, preferably dsDNA or a DNA origami structure, having between 500 and 3000 base pairs.

20. The method according to claim 1 wherein the sample is illuminated with a laser.

21. The method according to claim 1 wherein the sample comprises a body fluid from a human or non-human animal subject or a fluid from a plant.

22. A solid support, preferably a coverslip, glass microscope slide, optical fiber, prism, microtiter plate or microfluidic chip, having a first proximity probe tethered thereto by a polymeric or biopolymeric tether molecule having a mean end-to-end distance of about 50 nm to about 1000 nm in aqueous solution.

23. The solid support according to claim 22 wherein the tether molecule comprises a surfactant, lipid, peptide, protein, oligosaccharide, polysaccharide, oligonucleotide or polynucleotide.
